(19) United States
(12) Patent Application Publication
(10) Pub. No.: US 2001/0046950 A1
(43) Pub. Date: Nov. 29, 2001

(54) Process for preparing a detergent bar composition

(76) Inventors: Deepak Agrawal, Near Gumastha Nagar (IN); Rajapandian Benjamin, Whitefield (IN); Dhanraj Kalyansundaram Chokappa, Chakala (IN); Sudhakar Yeshwant Mhaskar, Chakala (IN)

Correspondence Address: UNILEVER, PATENT DEPARTMENT, 45 RIVER ROAD, EDGEWATER, NJ, 07020, US

(21) Appl. No.: 09/730,138

(22) Filed: Dec. 5, 2000

(30) Foreign Application Priority Data
Dec. 8, 1999 (IN) 904/BOM/99

(51) Int. Cl. A61K 7/50; C11D 7/02

(52) U.S. Cl. 510/141; 510/152; 510/155; 510/447; 510/507; 510/508

(57) ABSTRACT

A process for preparing a detergent bar composition comprising from 5 to 70% by weight fo detergent active, from 0.5 to 30% by weight of amorphous alumina, from 0.5 to 30% by weight of at least one akali metal salt of carboxylic acid, from 10 to 55% by weight of water, and 0-30% of detergent builder, which process comprises the steps of:

a) reacting one or more precursors of the detergent active and at least one carboxylic acid with an aluminium containing alkaline material to obtain a mixture of amorphous alumina, carboxylate and detergent active at a temperature between 25° C. to 95° C.;

b) adding any other actives or additives such as herein described to the mixture of step (a); and

c) converting the product into bars.

The invention relates to a process for the preparation of soap/detergent bars for personal/fabric washing or for hard surface cleaning. This invention particularly relates to an improved process for preparing low density detergent bar comprising high levels of water and other liquid benefit agents.

Conventional detergent bars, based on soap for personal washing contain over about 70% by weight total fatty matter (TFM), the remainder being water (about 10-15%) and other ingredients such as colour, perfume, preservatives, etc. Structurants and fillers are also present in such compositions in small amounts which replace some of the soap in the bar while retaining the desired hardness of the bar. A few known fillers include starch, kaolin and talc.

Hard non-milled soaps containing moisture of less than 35% are also available. These bars have a TFM of about 30-65%. The reduction in TFM has been achieved by the use of insoluble particulate materials and/or soluble silicates. Milled bars generally have a water content about 8-15% and the hard non-milled bars have a water content of about 20-35%.

Fabric washing compositions contain, as an essential ingredient, a surfactant system whose role is to assist in removal of soil from the fabric and its suspension in the wash liquor. Detergent bars require an acceptable physical strength so that they retain their structural integrity during handling, transport and use. The hardness of the bars, at the time of manufacture and subsequently, is an especially important property. Inclusion of certain ingredients to make the bar harder usually results in higher density bars, making the bars considerably smaller and thus less attractive to the consumer, and also being gritty to feel. Commercially available detergent bars contain detergent active components and detergent builders together with optional components such as for example abrasives, fillers, perfumes, alkaline salts and bleaching agents.

Commercial hard surface cleaning compositions typically comprise one or more surfactants and a plurality of abrasives dispersed therein. Combinations of these together with electrolytes are generally used to form a suspending system as is well known in the art.

Increased water structuring of the bar help in improving the in use properties of the bar without affecting its physical properties in an economical way. It enables one to manufacture detergent bars cost effectively. It is important to deliver sensory properties such as lather, cleaning, product feel and skin feel without altering the processability and physical properties of the bar, and to process the formulations using the existing equipment. This would enable products to be processed by the conventional methods of manufacture and without altering the through-put.

IN 177828 discloses a process wherein by providing a balanced combination of aluminium hydroxide and TFM it is possible to prepare a low TFM bar having high water content but with satisfactory hardness. The patent teaches the generation of colloidal alumina hydrate in-situ by a reaction of fatty acid with an aluminium containing alkaline material such as sodium aluminate to form bars which are obtained by plodding.

Our copending application 810/Bom/98 discloses a process of preparing a low TFM composition by a reaction of fatty acid/fat with an aluminium containing alkaline material such as sodium aluminate solution that specifically has a solid content of 20 to 55%, wherein the alumina (Al₂O₃) to sodium oxide (Na₂O) is in a ratio of 0.5 to 1.55:1 by weight to give superior bar properties. These bars have improved hardness and smoother feel. This reaction can take place in a broad temperature range of 40 to 95° C.

It has now been found that in-situ generation of amorphous alumina, preferably by a reaction of fatty acid/fat or an acid precursor of an active detergent in the presence of carboxylic acid with an equivalent weight less than 150 with an aluminium containing alkaline material such as sodium aluminate solution that specifically has a solid content of 20 to 55%, wherein the alumina (Al₂O₃) to sodium oxide (Na₂O) is in a ratio of 0.5 to 1.55 by weight, gives superior bar properties. These bars will be high moisture detergent compositions with good processability and improved water retention capacity.

Accordingly, this invention provides an improved process for preparing detergent bar composition comprising:

from 5 to 70% by weight of detergent active;

from 0.5 to 30% by weight of amorphous alumina;

from 0.5 to 30% by weight of at least one alkali metal salt of carboxylic acid;

from 10 to 55% by weight of water;

optionally other benefit agents; and

0-30% of detergent builder;

which process comprises the steps of:

a. reacting one or more precursors of detergent active and at least one carboxylic acid such as herein described with an aluminium containing alkaline material such as sodium aluminate with a solid content of 20 to 55%, wherein the Al₂O₃ to Na₂O is in a ratio of 0.5 to 1.55 by weight to obtain a mixture of amorphous alumina, carboxylate and detergent active at a temperature between 25° C. and 95° C.;

b. adding if desired, other detergent actives, builders and minor additives such as herein described to the mixture of step (a); and

c. converting the product into bars by conventional methods.

The carboxylic acid mentioned in step (a) are those which have an equivalent weight less than 150, and may be selected from aliphatic monocarboxylic acids that are not fatty acids, and their polymers. More preferably they are C₁ to C₅ carboxylic acids and their polymers. Other suitable carboxylic acids are aliphatic or aromatic di, -tri-, or polycarboxylic acids and hydroxy- and amino carboxylic acids.

It is preferred that the weight ratio of the precursor of detergent active to the carboxylic acid is in the range 1 to 60:1.

According to a preferred aspect, this invention provides an improved process for preparing detergent bar composition comprising:

from 5 to 70% by weight of soap or non-soap detergent active;

from 0.5 to 30% by weight of amorphous alumina;

from 0.5 to 30% by weight of at least one alkali metal salt of carboxylic acid;

from 10 to 55% by weight of water;

optionally other liquid benefit agents; and

0-30% of detergent builder,

which process comprises the steps of:

a. reacting at least one carboxylic acid such as herein described with an aluminium containing alkaline material such as sodium aluminate with a solid content of 20 to 55%, wherein the Al₂O₃ to Na₂O is in a ratio of 0.5 to 1.55 by weight, to obtain a mixture of amorphous alumina and carboxylate at a temperature between 25° C. and 95° C., followed by the addition of the precursor of the detergent active;

b. adding if desired, other detergent actives, builders and minor additives such as herein described to the mixture of step (a); and

c. converting the product into bars by conventional method.

The sodium aluminate in reaction (a) is at least equal to the stoichiometric amount required for the neutralisation of carboxylic acid and the precursor of detergent active.

According to another preferred aspect of this invention there is provided a process for preparing a low TFM detergent bar composition comprising:

from 15 to 70% by weight of total fatty matter;

from 0.5 to 30% by weight of amorphous alumina;

from 0.5 to 30% by weight of at least one alkali metal salt of carboxylic acid;

from 10 to 55% by weight of water;

optionally other liquid benefit agents; and

the balance being other and minor additives as herein described, which process comprises the steps of:

a. reacting a mixture of one or more fatty acids/fat and at least one carboxylic acid such as herein described with an aluminium containing alkaline material such as sodium aluminate with a solid content of 20 to 55%, wherein the Al₂O₃ to Na₂O is in a ratio of 0.5 to 1.55 by weight to obtain a mixture of amorphous alumina, carboxylate and soap at a temperature of between 25° C. and 95° C.;

b. adding if desired, other detergent actives and minor additives such as herein described to the mixture of step (a); and

c. converting the product of step (b) into bars by conventional methods.

According to yet another preferred aspect of this invention there is provided an improved process for preparing detergent bar composition comprising:

from 10 to 70% by weight of non-soap detergent active;

from 0.5 to 30% by weight of amorphous alumina;

from 0.5 to 30% by weight of at least one alkali metal salt of carboxylic acid;

from 10 to 35% by weight of water;

optionally other liquid benefit agents;

0-60% inorganic particulates;

0-30% of detergent builder; and

1-15% sodium aluminosilicate;

which process comprises the steps of:

a. reacting at least one carboxylic acid such as herein described with an aluminium containing alkaline material such as sodium aluminate with a solid content of 20 to 55%, wherein the Al₂O₃ to Na₂O is in a ratio of 0.5 to 1.55 by weight to obtain a mixture of amorphous alumina and carboxylate at a temperature between 25° C. and 95° C., followed by the addition of the precursor of the non-soap detergent active;

b. in situ generation of sodium aluminosilicate by reacting sodium silicate with aluminium sulphate;

c. adding if desired, other detergent actives, builders, inorganic particulates and minor additives such as herein described to the mixture of step (b); and

d. converting the product of step (c) into bars by conventional methods.

The invention is carried out in any mixer conventionally used in soap/detergent manufacture, and is preferably a high-shear kneading mixer. The preferred mixers include a ploughshare mixer, mixers with kneading members of the Sigma type, multi-wiping overlap, single curve or double arm. The double arm kneading mixers can be of overlapping or tangential in design. Alternatively, the invention can be carried out in a helical screw agitator vessel, or multi-head dosing pump/high shear mixer and spray drier combinations as in conventional processing.

The carboxylic acids may be selected from monocarboxylic acids such as acetic acid, propionic acid, butanoic acid, isobutyric acid, etc., di/poly carboxylic acids such as succinic, malonic, malic, maleic, citric and tartaric acid etc. or their polymers such as polyacrylic acids, acrylic—maleic copolymers, etc.

Hydroxy carboxylic acids selected from glycolic, lactic, ricinoleic, or the amino carboxylic acids selected e.g. from glycine, valine, and leucine may also be employed.

The detergent active used in the process may be soap or non-soap surfactants. The term total fatty matter, usually abbreviated to TFM, is used to denote the percentage by weight of fatty acid and triglyceride residues present in soaps without taking into account the accompanying cations.

For a soap having 18 carbon atoms, an accompanying sodium cation will generally amount to about 8% by weight Other cations may be employed as desired, such as for example zinc, potassium, magnesium, alkyl ammonium and aluminium.

The term soap denotes salts of carboxylic fatty acids. The soap may be derived from any of the triglycerides conventionally used in soap manufacture—consequently the carboxylate anions in the soap may typically contain from 8 to 22 carbon atoms.

The soap may be obtained by saponifying a fat and/or a fatty acid. The fats or oils generally used in soap manufacture may be such as tallow, tallow stearines, palm oil, palm stearines, soya bean oil, fish oil, caster oil, rice bran oil, sunflower oil, coconut oil, babassu oil, palm kernel oil, and others. In the above process, the fatty acids are derived from oils/fats selected from coconut, rice bran, groundnut, tallow, palm, palm kernel, cotton seed, soybean, castor etc. The fatty acid soaps can also be synthetically prepared (e.g. by the oxidation of petroleum, or by the hydrogenation of carbon monoxide by the Fischer-Tropsch process). Resin acids, such as those present in tall oil, may be used. Naphthenic acids are also suitable.

Tallow fatty acids can be derived from various animal sources, and generally comprise about 1-8% myristic acid, about 21-32% palmitic acid, about 14-31% stearic acid, about 0-4% palmitoleic acid, about 36-50% oleic acid and about 0-5% linoleic acid. A typical distribution is 2.5% myristic acid, 29% palmitic acid, 23% stearic acid, 2% palmitoleic acid, 41.5% oleic acid, and 3% linoleic acid. Other similar mixtures, such as those from palm oil and those derived from various animal tallow and lard are also included.

Coconut oil refers to fatty acid mixtures having an approximate carbon chain length distribution of 8% C₈, 7% C₁₀, 48% C₁₂, 17% C₁₄, 8% C₁₆, 2% C₁₈, 7% oleic and 2% linoleic acids (the first six fatty acids listed being saturated). Other sources having similar carbon chain length distributions, such as palm kernel oil and babassu kernel oil, are included within the term “coconut oil”.

A typical fatty acid blend consisted of 5 to 30% coconut fatty acids and 70 to 95% fatty acids ex. hardened rice bran oil. Fatty acids derived from other suitable oils/fats such as groundnut, soybean, tallow, palm, palm kernel, etc. may also be used in other desired proportions.

The composition according to the invention will also preferably comprise detergent actives which are generally chosen from both anionic and nonionic detergent actives.

Suitable anionic detergent active compounds are water soluble salts of organic sulphuric reaction products having in the molecular structure an alkyl radical containing from 8 to 22 carbon atoms, and a radical chosen from sulphonic acid or sulphuric acid ester radicals and mixtures thereof.

Examples of suitable anionic detergents are sodium and potassium alcohol sulphates, especially those obtained by sulphating the higher alcohols produced by reducing the glycerides of tallow or coconut oil; sodium and potassium alkyl benzene sulphonates such as those in which the alkyl group contains from 9 to 15 carbon atoms; sodium alkyl glyceryl ether sulphates, especially those ethers of the higher alcohols derived from tallow and coconut oil; sodium coconut oil fatty acid monoglyceride sulphates; sodium and potassium salts of sulphuric acid esters of the reaction product of one mole of a higher fatty alcohol and from 1 to 6 moles of ethylene oxide; sodium and potassium salts of alkyl phenol ethylene oxide ether sulphate with from 1 to 8 units of ethylene oxide molecule and in which the alkyl radicals contain from 4 to 14 carbon atoms; the reaction product of fatty acids esterified with isethionic acid and neutralised with sodium hydroxide where, for example, the fatty acids are derived from coconut oil and mixtures thereof.

The preferred water-soluble synthetic anionic detergent active compounds are the alkali metal (such as sodium and potassium) and alkaline earth metal (such as calcium and magnesium) salts of higher alkyl benzene sulphonates and mixtures with olefin sulphonates and higher alkyl sulphates, and the higher fatty acid monoglyceride sulphates.

Suitable nonionic detergent active compounds can be broadly described as compounds produced by the condensation of alkylene oxide groups, which are hydrophilic in nature, with an organic hydrophobic compound which may be aliphatic or alkyl aromatic in nature. The length of the hydrophilic or polyoxyalkylene radical which is condensed with any particular hydrophobic group can be readily adjusted to yield a water-soluble compound having the desired degree of balance between hydrophilic and hydrophobic elements.

Particular examples include the condensation product of aliphatic alcohols having from 8 to 22 carbon atoms in either straight or branched chain configuration with ethylene oxide, such as a coconut oil ethylene oxide condensate having from 2 to 15 moles of ethylene oxide per mole of coconut alcohol; condensates of alkylphenols whose alkyl group contains from 6 to 12 carbon atoms with 5 to 25 moles of ethylene oxide per mole of alkylphenol; condensates of the reaction product of ethylenediamine and propylene oxide with ethylene oxide, the condensate containing from 40 to 80% of polyoxyethylene radicals by weight and having a molecular weight of from 5,000 to 11,000; tertiary amine oxides of structure R₃NO, where one group R is an alkyl group of 8 to 18 carbon atoms and the others are each methyl, ethyl or hydroxyethyl groups, for instance dimethyldodecylamine oxide; tertiary phosphine oxides of structure R₃PO, where one group R is an alkyl group of from 10 to 18 carbon atoms, and the others are each alkyl or hydroxyalkyl groups of 1 to 3 carbon atoms, for instance dimethyldodecylphosphine oxide; and dialkyl sulphoxides of structure R₂SO where the group R is an alkyl group of from 10 to 18 carbon atoms and the other is methyl or ethyl, for instance methyltetradecyl sulphoxide; fatty acid alkylolamides; alkylene oxide condensates of fatty acid alkylolamides and alkyl mercaptans.

It is also possible to include cationic, amphoteric, or zwitterionic detergent actives in the compositions according to the invention.

Suitable cationic detergent actives that can be incorporated are alkyl substituted quarternary ammonium halide salts e.g. bis (hydrogenated tallow) dimethylammonium chlorides, cetyltrimethyl ammonium bromide, benzalkonium chlorides and dodecylmethylpolyoxyehtylene ammonium chloride and amine and imidazoline salts for e.g. primary,secondary and tertiary amine hydrochlorides and imidazoline hydrochlorides.

Suitable amphoteric detergent-active compounds that optionally can be employed are derivatives of aliphatic secondary and tertiary amines containing an alkyl group of 8 to 18 carbon atoms and an aliphatic radical substituted by an anionic water-solubilizing group, for instance sodium 3-dodecylamino-propionate, sodium 3-dodecylaminopropane sulphonate and sodium N-2-hydroxydodecyl-N-methyltaurate.

Suitable zwitterionic detergent-active compounds that optionally can be employed are derivatives of aliphatic quaternary ammonium, sulphonium and phosphonium compounds having an aliphatic radical of from 8 to 18 carbon atoms and an aliphatic radical substituted by an anionic water-solubilising group, for instance 3-(N-N-dimethyl-N-hexadecylammonium) propane-1-sulphonate betaine, 3-(dodecylmethyl sulphonium) propane-l-sulphonate betaine and 3-(cetylmethylphosphonium) ethane sulphonate betaine.

It is especially preferred for personal wash systems of the invention to include up to 30% of other liquid benefit agents such as non-soap surfactants, skin benefit materials such as moisturisers, emollients, sunscreens, and anti-ageing compounds. These are incorporated at any step prior to step of milling. Alternatively certain of these benefit agents can be introduced as macro domains during plodding.

For the purpose of the invention, the alkaline material used is sodium aluminate preferably with a solid content of 20 to 55%, preferably wherein the Al₂O₃ to Na₂O is in a ratio of 0.5 to 1.55 by weight. However the specified Al₂O₃ to Na₂O ratio is preferably 1.0 to 1.5.

The detergency builders which may be used in the formulation are preferably inorganic and suitable builders include, for example, alkali metal aluminosilicates (zeolites), alkali metal carbonate, sodium tripolyphosphate (STPP), tetrasodium pyrophosphate (TSPP), citrates, sodium nitrilotriacetate (NTA) and combinations of these. Builders are suitably used in an amount ranging from 1 to 30% by wt.

Examples of useful and suitable moisturisers and humectants include polyols, glycerol, cetyl alcohol, carbopol 934, ethoxylated castor oil, paraffin oils, lanolin and its derivatives. Silicone compounds such as silicone surfactants like DC3225C (Dow Corning) and/or silicone emollients, silicone oil (DC-200 Ex-Dow Corning) may also be included. Sun-screens such as 4-tertiary butyl-4′-methoxy dibenzoylmethane (available under the trade name PARSOL 1789 from Givaudan) and/or 2-ethyl hexyl methoxy cinnamate (available under the trade name PARSOL MCX from Givaudan) or other UV-A and UV-B sun-screens. Water soluble glycols such as propylene glycol, ethylene glycol, glycerol, may be employed at levels up to 10%.

An inorganic particulate phase is not an essential ingredient of the formulation, but may be incorporated, especially for hard surface cleaning compositions. Preferably, the particulate phase comprises a particulate structurant and/or abrasive which is insoluble in water. In the alternative, the abrasive may be soluble and present in such excess to any water present in the composition that the solubility of the abrasive in the aqueous phase is exceeded, and consequently solid abrasive exists in the composition.

Suitable inorganic particulates can be selected from, particulate zeolites, calcites, dolomites, feldspars, silicas, silicates, other carbonates, bicarbonates, borates, sulphates and polymeric materials such as polyethylene.

The most preferred inorganic particulates are calcium carbonate (as e.g. Calcite), mixtures of calcium and magnesium carbonates (as e.g. dolomite), sodium hydrogen carbonate, borax, sodium/potassium sulphate, zeolite, feldspars, talc, koalin and silica.

Calcite, talc, kaolin, feldspar and dolomite and mixtures thereof are particularly preferred, due to their low cost and colour.

The inorganic particulate structurants such as alumino silicate may be generated in situ using aluminium sulphate and sodium silicate in the formulation. It is also possible to incorporate readily available sodium alumino-silicate into the formulation.

Other additives such as one or more water insoluble particulate materials such as e.g. talc or kaolin, polysaccharides such as starch or modified starches and celluloses may be incorporated.

In step (b) of the process, minor and conventional ingredients preferably selected from enzymes, antiredeposition agents, fluorescers, colour, preservatives and perfumes, also bleaches, bleach precursors, bleach stabilisers, sequestrants, soil release agents (usually polymers) and other polymers may optionally be incorporated at a level of up to 10 wt %.

Illustrations of a few non-limiting examples by way of demonstration only are provided herein showing comparative results of the composition prepared both within the present invention and beyond the invention, with reference to

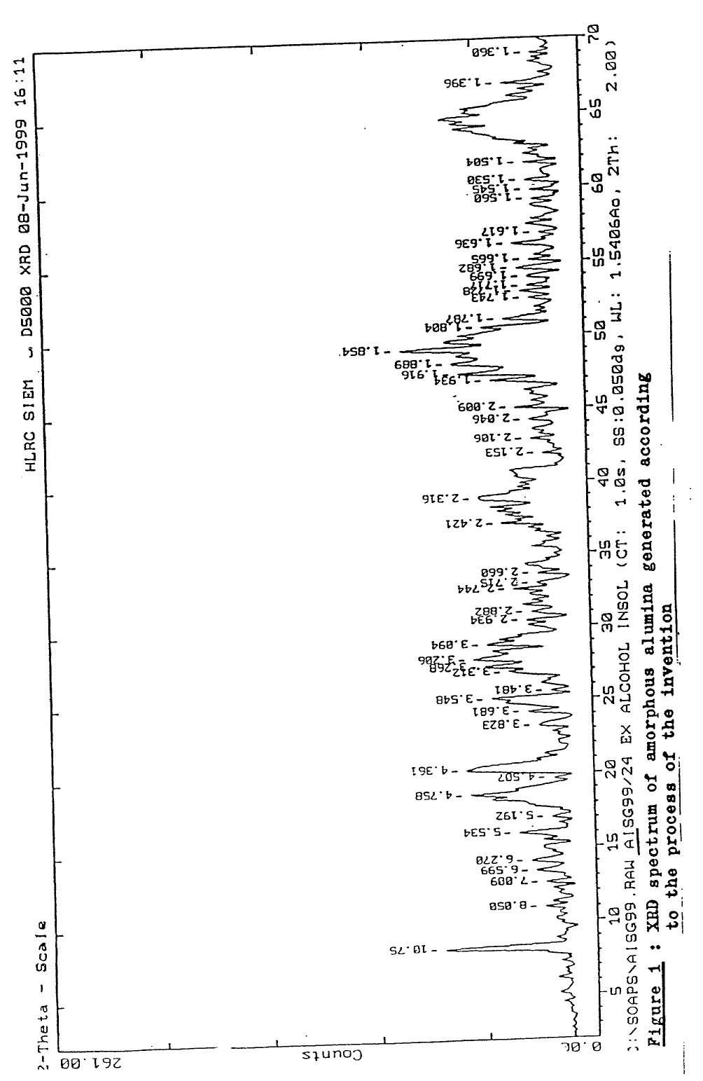

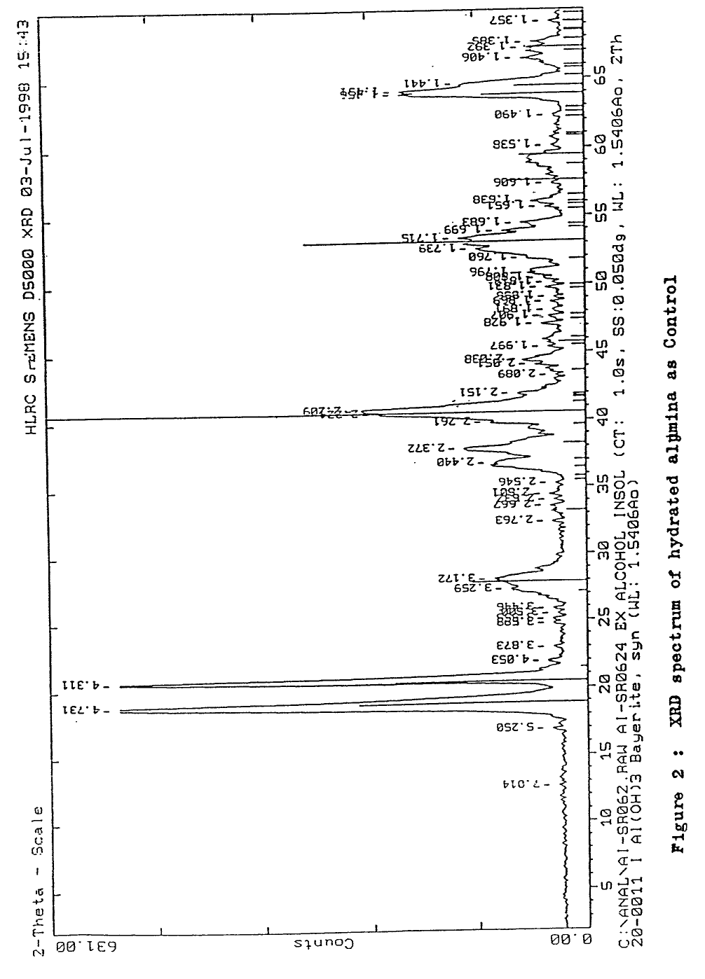

FIGS. 1 and 2, which show x ray diffraction spectra of samples generated according to the examples.

### EXAMPLES

Illustrations of a few non-limiting examples are provided herein showing comparative results of the composition prepared by the present invention and with aluminium hydroxide.

Process for Preparing the Soap Bar:

a. Conventional Process

### Example 1 to 3

A batch of 50 kg soap was prepared by melting a mixture of fatty acids at 80-85° C. in a crucher and neutralising with 48% sodium hydroxide solution in water. Additional water was added to obtain the moisture content of about 33%. The soap mass was spray dried under vacuum and formed into noodles. The soap noodles were mixed with soda ash, talc, perfume, colour, and titanium dioxide in a sigma mixer, and passed twice through a triple roll mill. The milled chips were plodded under vacuum and formed into billets. The billets were cut and stamped into tablets.

b. Process of Generation of Hydrated Alumina

### Example 4

A batch of 50 kg soap was prepared by melting a mixture of fatty acids at 80-85° C. in a sigma mixer and neutralising with 40% sodium aluminate solution. The sodium aluminate solution was prepared by dissolving solid alumina trihydrate in sodium hydroxide solution at 90-95° C. The soap was cooled to about to 35° C. and mixed with soda ash, perfume, colour, and titanium dioxide. The soap was then passed twice through a triple roll mill. The milled chips were plodded under vacuum and formed into billets. The billets were cut and stamped into tablets.

### c. Process According to the Invention

### Example 5 & 6

A batch of 50 kg soap was prepared by melting a mixture of fatty acids and citric acid (Example 5) and along with linear alkyl benzene sulphonic acid (LAS) (Example 6) at 80-85° C. in a sigma mixer and neutralising with 40% sodium aluminate solution. The sodium aluminate solution was prepared by dissolving solid alumina trihydrate in sodium hydroxide solution at 90-95° C. The soap was cooled to about to 35° C. and mixed with soda ash, perfume, colour, titanium dioxide and other conventional additives. The soap was then passed twice through a triple roll mill. The milled chips were plodded under vacuum and formed into billets. The billets were cut and stamped into tablets.

The samples prepared as described above were tested for hardness and in use properties such as water retention, hardness and feel by the following procedure.

Water Retention:

The bars were weighed and stored at room temperature 25-30° C. for 90 days. The weight of the bars was taken periodically up to 90 days. The data is presented as % water retained in the bar at the end of 90 days.

Yield Stress:

Yield stress quantifies the hardness of a soap bar. The yield stress of the bars at a specified temperature was determined by observation of the extent to which a bar was cut by a weighted cheese wire during a specified time. The apparatus consists of a cheesewire (diameter d in cm) attached to a counter balanced arm which can pivot freely via a ball race bearing. A billet of soap is positioned under the wire such that the wire is just in contact with one edge of the billet. By applying a weight (W g.) directly above the cheesewire, a constant force is exerted on the wire which will slice into the soap. The area over which the force acts will increase as the depth of cut increases and therefore the stress being exerted will decrease until it is exactly balanced by resistance of the soap and the wire stops moving. The stress at this point is equal to the yield stress of the soap.

The time taken to reach this point was found to be 30 secs. so that a standard time of 1 min was chosen to ensure that the yield stress had been reached. After this time the weight was removed and the length of the cut (L in Cm) measured. The yield stress is calculated using the semi-empirical formula:

$$Y . S = \frac{3 W}{8} \times \frac{98.1}{L \times d} \text{Pascal (Pa.)}$$

In terms of bar feel, a standard washing procedure in cold water is followed for estimation of grittiness by feel by a group of trained panellists. The score is given over scale of 1-10, where score of 1 relates to the best feel and 10 to the poorest. The toilet soaps with acceptable quality generally have a feel score in the range of 7.8 to 8.0.

TABLE 1

| Composition % wt. | Ex 1 | Ex 2 | Ex 3 | Ex 4 | Ex 5 | Ex 6 |
|---|---|---|---|---|---|---|
| TFM-Na | 73.4 | 68 | 50 | 66 | 55 | 48 |
| Amorphous alumina | — | — | — | — | 12 | 14 |
| Carboxylic-Na | — | — | — | — | 12 | 12 |
| Non-soap active | — | — | — | — | — | 4 |
| (LAS Na) | | | | | | |
| Hydrated Alumina | — | — | — | 13.5 | — | — |
| Talc | 11 | 11 | 29 | — | — | — |
| Minor ingredients | 3.1 | 3 | 3 | 3 | 3 | 3 |
| Water | 12.5 | 18 | 18 | 17.5 | 18 | 19 |
| Bar Properties | | | | | | |
| % water after 90 | 7.5 | 7.5 | 7.0 | 8.3 | 10.1 | 12.81 |
| days (at RT) | | | | | | |
| Hardness (Pa) | 2.9 | 1.2 | 2.8 | 2.8 | 2.8 | 2.8 |
| Feel | 8.3 | 8.3 | 8.3 | 7.7 | 7.7 | 7.7 |

The data presented in Table 1 show that when the bar is formulated with a conventional material such as talc the level of water that can be incorporated is only up to about 13%. If the water level is increased, the bars become un-processable. The in use properties such as feel are inferior as compared to the high TFM soaps. The bars prepared according to the invention structure higher levels of water and/or other liquid benefit agents, have good physical properties and also retain a higher percentage of this water during storage as compared to any of the conventional bars described above, whilst maintaining the sensory properties of high TFM soaps.

Non-soap Detergent Bar Processing

Control Process

A 10 kg batch was prepared. Several control formulations and a formulation according to the invention were processed using the compositions as indicated in Table 2. Linear alkyl benzene sulphonate (LAS) was taken in a sigma mixer and neutralised using sodium carbonate (Example 7). Other ingredients such as builders, inorganic particulates, other conventional ingredients, water etc. were mixed and plodded by the conventional route. A mixture of LAS and citric acid (Example 8) was taken in the mixer and the same procedure was followed. In Example 9 LAS was neutralised with sodium aluminate instead of sodium carbonate, and in Example 10 this was followed by the addition of citric acid, and the rest of the procedure was as described for Example 7.

Process According to the Invention

Amorphous alumina—carboxylate and detergent active was generated by a reaction of a mixture of LAS and citric acid with sodium aluminate with 44% solids content, having an Al₂O₃ to Na₂O ratio of 1.1 by weight in a sigma mixer (Example 11). The other ingredients as indicated in Table 2 were added and mixed. The dough was converted into bars by the conventional route.

The bars were analysed by the following procedure.

Penetration Value (PV)

Penetration value indicating the hardness of the bar was measured using a cone penetrometer; the details of a typical instrument and the method of measurement is given below.

Cone type Penetrometer

MANUFACTURER: Adair Dutt & Company, Bombay.

RANGE OF MEASUREMENT: 0-40 mm

RANGE OF VERIFICATION: 20 in steps of 5

Procedure of Measurement: Let the entire mass (comprised of penetrometer needle and standard weight) which just rests on the test sample drop freely and thus penetrate the test mass to a specific distance for a specified period of time, and read of this distance to 1/10^{th} of mm. Take the average after repeating the exercise for at least 3 times.

Density of the Bar:

The density of the bar is measured by the standard method and calculated using the formula:

$$\text{Density} ( grams / {cm}^{3} ) = \frac{\text{Weight of bar (grams)}}{\text{Volume in} {cm}^{3}}$$

Water Activity:

Water activity is a measure of water loss encountered during storage from a substance. It is expressed in terms of relative humidity (%) and was measured using a water activity meter (e.g. TH 200 Thermo constanter from Novasina).

TABLE 2

| Composition % wt. | Ex 7 | Ex 8 | Ex 9 | Ex 10 | Ex 11 |
|---|---|---|---|---|---|
| Detergent active | 21 | 21 | 21 | 21 | 21 |
| (LAS) | | | | | |
| Citric acid | — | 2 | — | 2 | 2 |
| Hydrated alumina | — | — | 6.4 | 6.4 | — |
| Amorphous alumina | — | — | — | — | 9.5 |
| Builders (STPP, Soda) | 20 | 20 | 20 | 20 | 20 |
| Calcite | 36.44 | 34 | 24.5 | 22.5 | 16 |
| China clay | 8.33 | 8.33 | 8.33 | 8.33 | 8.33 |
| Sodium | 4.0 | 4.0 | 4.0 | 4.0 | 4.0 |
| aluminosilicate | | | | | |
| Conventional | 3.7 | 3.7 | 3.7 | 3.7 | 3.7 |
| ingredients | | | | | |
| Water | To 100 | To 100 | To 100 | To 100 | To 100 |
| Product | | | | | |
| Characteristics | | | | | |
| Penetration Value | 20 | 20 | 25 | 25 | 25 |
| Water activity % | 82 | 82 | 88 | 88 | 78 |
| Density | 1.9 | 1.9 | 1.75 | 1.75 | 1.6 |

Data presented in Table 2 show that incorporation of amorphous alumina in the formulation gives a synergistic benefit as compared to use citric acid or hydrated alumina alone or in combination in the formulation.

Characterisation of the Amorphous Alumina

The sample of amorphous alumina generated by melting a mixture of fatty acids and citric acid along with linear alkyl benzene sulphonic acid (LAS) (Example 6) at 80-85° C. in a sigma mixer, and neutralising with 40% sodium aluminate and a hydrated alumina generated by melting a mixture of fatty acids at 80-85° C. in a sigma mixer and neutralising with 40% sodium aluminate solution (Example 4) has been analysed for crystallinity. The XRD spectrum has been recorded for 2θ ranging from 0-70°. The sample has been scanned at 0.50 per second. The XRD spectrum recorded for amorphous alumina has been presented in FIG. 1 and that for hydrated alumina in FIG. 2. The absence of any distinct peaks in the spectrum presented in FIG. 1 shows the amorphous nature of the alumina generated by the process according to the invention in comparison to hydrated alumina (control) presented in FIG. 2 which shows distinct peaks indicating crystalline nature.

1. A process for preparing a detergent bar composition comprising from 5 to 70% by weight of detergent active, from 0.5 to 30% by weight of amorphous alumina, from 0.5 to 30% by weight of at least one akali metal salt of carboxylic acid, from 10 to 55% by weight of water, and 0-30% of detergent builder, which process comprises the steps of:
a) reacting one or more precursors of the detergent active and at least one carboxylic acid with an aluminium containing alkaline material to obtain a mixture of amorphous alumina, carboxylate and detergent active at a temperature between 25° C. and 95° C.;
b) adding any other actives or additives as described to the mixture of step (a); and
c) converting the product into bars.

2. A process according to claim 1, wherein the aluminium containing alkaline material is sodium aluminate with a solid content of 20-55%, and wherein Al₂O₃ to Na₂O ratio is in the region 0.5 to 1.55:1.

3. A process according to any of claims 1 or 2, wherein the carboxylic acid has an equivalent weight of less than 150.

4. A process according to claim 3 wherein the carboxylic acid is a monocarboxylic acid which is not a fatty acid.

5. A process according to claim 4 wherein the carboxylic acid is acetic acid, propionic acid, butanoic acid, isobutyric acid, succinic acid, malonic acid, malic acid, maleic acid, citric acid, tartaric acid, glycolic acid, lactic acid, ricinoleic acid, or an amino carboxylic acid of glycine, valine or leucine, or mixtures thereof.

6. A process according to any of the proceeding claims, wherein the weight ratio of the precursor detergent active to carboxylic acid is in the range 1 to 60:1.

7. A process according to any of the proceeding claims, wherein the detergent active is fatty matter and the result bar is a low total fatty matter detergent bar.

8. A process according to any of the proceeding claims, wherein the composition additionally comprises up to 60% of inorganic particulate.

9. A process according to any of the proceeding claims, wherein the composition additionally comprises up to 30% of a detergency builder.

10. A process according to any of the proceeding claims, wherein the composition additionally comprises 1 to 15% of sodium aluminosilicate.

11. A process according to any of the proceeding claims, wherein the process includes the in situ generation of sodium aluminosilicate by the reaction of sodium silicate with aluminium sulphate.
